(19) United States
(12) Patent Application Publication
(10) Pub. No.: US 2011/0256605 A1
(43) Pub. Date: Oct. 20, 2011

(54) CELLS WITH NON-NATURAL PHYSIOLOGIES DERIVED BY EXPRESSING LIGHT-POWERED PROTON PUMPS IN ONE OR MORE MEMBRANES

(75) Inventors: Jan T. Liphardt, Berkeley, CA (US); Jessica M. Walter, Albany, CA (US); Jasper Rine, Richmond, CA (US); Carlos Bustamante, Berkeley, CA (US)

(73) Assignee: The Regents of the University of California, a California corporation, Oakland, CA (US)

(21) Appl. No.: 13/044,728

(22) Filed: Mar. 10, 2011

Related U.S. Application Data
(60) Provisional application No. 61/313,615, filed on Mar. 12, 2010.

(51) Int. Cl. C12P 1/00; C12N 1/21; C12N 13/00; C12M 1/42; C12P 1/04; C12P 1/02; C12N 1/00; C12N 1/19

(52) U.S. Cl. 435/170; 435/243; 435/252.33; 435/254.21; 435/289.1; 435/41; 435/171; 435/173.1

(57) ABSTRACT

Methods and procedures for designing and constructing microbes with the ability to harvest light energy are described. In certain embodiments, these methods and procedures are used to construct a photosynthetic yeast based on proteorhodopsin (PR) expression. Proteorhodopsin is a light powered proton pump used by some ocean bacteria to scavenge light energy. By illuminating single yeast cells expressing PR, controlled amounts of energy can be delivered to these cells. A light-harvesting yeast is a unique bioenergetics research platform for investigating the interplay of biofuel production, cellular ATP levels, and the proton-motive-force (pmf). Also, a strain of yeast with light-boosted biomass to biofuel conversion efficiency possesses direct industrial and commercial utility.

### CROSS-REFERENCES TO RELATED APPLICATIONS

This application claims priority to U.S. Provisional Application No. 61/313,615, filed Mar. 12, 2010, which is incorporated in its entirety herein for all purposes.

### STATEMENT AS TO RIGHTS TO INVENTIONS MADE UNDER FEDERALLY SPONSORED RESEARCH OR DEVELOPMENT

This work was supported in part by Contract No. DE-AC02-05CH11231 awarded by the US Department of Energy. The U.S. government has certain rights in the invention.

### BACKGROUND OF THE INVENTION

A major goal of bioenergy research is to increase the efficiency with which microbes convert biomass into fuels such as butanol. Accomplishing this task requires fundamental studies of cellular energy and metabolite fluxes; analyses of the whole genome transcriptome, the proteome, and metabolome; and modeling and re-engineering of various microbial subsystems (e.g., energy harvesting, sugar uptake, and fermentation pathways). Generally speaking, the biofuel production efficiency (the ratio of input biomass to output biofuel) of a given microbe can be limited by factors including the unsuitability of the input biomass, insufficient cellular energy, and the toxicity of inputs, intermediates, or products. A microbe, e.g., a yeast, with increased biomass to biofuel conversion efficiency would be of great industrial and commercial utility.

### BRIEF SUMMARY OF THE INVENTION

In one aspect, the present invention provides a photosynthetic yeast based on recombinant proteorhodopsin (PR) expression. Proteorhodopsin is a light powered proton pump used by some ocean bacteria to scavenge light energy. By illuminating single yeast cells expressing PR, it is possible to deliver controlled amounts of energy to these cells and then watch how these cells use this extra energy.

The light-harvesting yeast described herein provides a unique bioenergetics research platform for investigating the interplay of biofuel production, cellular ATP levels, and the proton-motive-force (pmf). In addition, a strain of yeast whose internal energy fluxes can be externally boosted provides a source of additional energy for fermentation as well as other processes that can be manipulated for, e.g., industrial purposes.

Numerous prokaryotic and eukaryotic cells use light-powered proton pumps to harvest light energy. Well-characterized examples include Bacteriorhodopsin (occurring in the halobacteria) and Proteorhodopsin (PR, found in proteobacteria). Different light powered proton pumps feature different membrane requirements, different action spectra (i.e. color sensitivities), and different light collection efficiencies. Typically, the light-powered proton pump occurs in one cell membrane (such as the plasma membrane), allowing the cell to create and maintain a proton-motive force across that membrane.

Proton-motive forces are a central energy store of the cell. Once generated, a proton-motive force (pmf) can be used by the cell to perform a multitude of reactions of interest to medicine, energy, industry, and defense.

One such reaction consists of using the energy stored in the pmf to synthesize energy-rich molecules such as adenosine tri-phosphate (ATP), which the cell can use to convert biomass into biofuels.

Another such reaction consists of using the pmf generated by light-capture to directly power molecular machines such as the flagellar motor, yielding cells that swim when illuminated.

Another such reaction consists of using the pmf generated by light-capture to assist the cell in transporting metabolites across membranes such as the plasma membrane, decreasing the amount of energy the cell must divert from anabolism to take-up or extrude metabolites from/into the medium.

Another such reaction consists of using the ATP generated by light-capture to power ABC superfamily drug transporters, yielding cells that resist certain drugs if and only if illuminated with particular colors of light, some of which may penetrate deep inside tissues.

By choosing which combination of light-powered proton pumps to express in which combination of cell membranes, a variety of organisms can be produced with cell physiologies unlike those found in nature.

For example, in one embodiment, the invention provides a strain of yeast (or other microbe) with increased biomass to ethanol conversion efficiency. In a related embodiment, the invention provides a method of generating such a strain of yeast (or other microbe) by targeting PR to both the inner mitochondrial membrane and the plasma membrane. In addition (or alternatively), by targeting PR to both the inner mitochondrial membrane and the vacuole, a strain of yeast (or other microbe) can be synthesized featuring both increased growth in oxygen-poor conditions and light-controlled drug resistance. In related embodiments, the yeast strain (or other microbe) comprises a gene encoding a heterologous light-powered proton pump (e.g., PR) that has been modified to reduce its rate of translation relative to an unmodified version of the gene. In certain embodiments, the proportion of properly folded, membrane-inserted light-powered proton pumps to improperly folded proton pumps in the yeast strain (or other microbe) is increased relative to the proportion comprised by an otherwise identical yeast strain (or other microbe) expressing an unmodified version of the gene encoding the proton pump. In yet another related embodiment, where the heterologous proton pump is proteorhodopsin, the immediate C-terminal region of the native proteorhodopsin sequence is deleted.

In certain embodiments, methods and compositions for expressing the light-powered proton pump proteorhodopsin in yeast are provided that result in the spontaneous emergence in yeast of layered membrane stacks, reminiscent of chloroplasts in plants.

In another embodiment, the invention provides a system for effecting light-controlled drug-resistance, comprising (a) a light-collecting entity (e.g., a yeast strain such as that described above), wherein the entity comprises a light powered proton pump in one or more cellular compartments; and (b) a light-source. In a related embodiment, the cellular compartment(s) are selected from the group consisting of the vacuole, the ER, Golgi apparatus, and plasma membrane.

Proper function and control of the organisms described above requires at least (a) efficient targeting of light powered proton pumps to particular cell membranes and (b) simultaneous modulation of the cell's natural pmf generating and modulating reactions. The embodiments above and other embodiments are described in more detail in the following sections.

In another embodiment, the invention provides a method for enhancing the ability of a strain of yeast (or other microbe) to pump protons in response to illumination, comprising expressing in the microbe at least one gene encoding a heterologous light-powered proton pump, said gene having been modified to promote the formation of a membrane structure having a plurality of layers of a membrane in said light-harvesting entity. In a related embodiment, said heterologous light-powered proton pump comprises a proteorhodopsin polypeptide fused to a non-proteorhodopsin polypeptide that promotes protein dimerization or oligomerization. In some embodiments, said non-proteorhodopsin polypeptide comprises a nonmonomeric fluorescent protein tag. In some embodiments, said non-proteorhodopsin polypeptide comprises a nonmonomeric green fluorescent protein (GFP) tag.

### BRIEF DESCRIPTION OF THE DRAWINGS

FIG. 1 shows proteorhodopsin-containing (PR+) E. coli responding to light. (A) Overview of spectra and spectral overlaps. The PR absorption spectrum is shown with bars indicating illumination wavelengths (the bar to the left is green light, the bar to the right is red light). (B) Single PR⁺ bacterium swims faster when illuminated with green light. The cell's position is recorded at a rate of 0.5 Hz via constant red dark-field illumination throughout the entire track. Illumination with green light at the absorption peak is periodic (20s on, 20s off) and occurs only during the circle portion of the path (triangles show position when green light is off). Velocity during periods of green illumination (10.6±0.9 μm/s) is 96% higher than during periods of red illumination alone (5.4±0.4 μm/s, p<0.005, one-tailed t-test). Respiration has been inhibited with 30 mM azide in motility buffer. The inset panel shows raw tiled movie frames of swimming cell.

FIG. 2 shows the flagellar response to green light measured in PR⁺ and PR⁻ bacteria at different levels of respiratory inhibition. (A) Movie frames showing a tethered cell in red light and green light, a plot of the angular position of a cell versus time in 60 mM azide, and a schematic of tethered cell geometry. In red light, the cell rotationally diffuses about its attachment point. Green light (shaded area) leads to counter clockwise rotation of the cell. Cells without (B) and with (C)PR. Solid, dotted, and dashed lines show three representative cells per condition. Cell-to-cell variation in absolute rotation rates is due to variation of cell length and tethering geometry. With or without azide, cells lacking PR show no response to green light. Removal of oxygen also leads to light-responsiveness. At high azide concentrations (C, top), angular velocity is nearly zero until cells are illuminated with green light.

FIG. 3 shows how the response of PR⁺ bacteria to green light increases with respiratory inhibition and light intensity. (A) Benefit of illumination versus degree of inhibition of the respiratory system. The difference between angular velocity in green light and red light (ω_{G}-ω_{R}) becomes pronounced in PR⁺ bacteria (solid circles) as respiration is inhibited by low oxygen or sodium azide. PR⁻ cells (hollow circles) show no change between red and green illumination. To facilitate comparison between cells, the angular velocities are normalized by each bacterium's maximum velocity. N=5 to 14 cells per condition. Green line: fit to model described in panel D. (B) The rotation speed of PR⁺ cells depends on the intensity of green illumination. Individual PR⁺ spinner cells were exposed to six intensities of green light. The mean angular velocity at each intensity is plotted (N=5 to 6 cells for each intensity), normalized by the velocity at maximum illumination. Dashed lines: fits to model described in D. Error bars are s.e.m. (C) Overview of transmembrane fluxes and proton pumping in PR⁺ E. coli. Sources of proton motive force include respiration. Sinks include rotation of the flagellar motor and ATP synthesis. (D) Model including sources of pmf (respiration, proteorhodopsin), sinks (such as the flagellar motor), and the membrane capacitance. The variable resistors R_{R} and R_{PR} model the effect of azide and light on proton extrusion by respiration and PR, respectively. The voltmeter (top-most circuit element) measures the potential difference across the membrane (equivalent to the pmf).

FIG. 4a shows the rotation rate of flagella-bound bacteria versus azide concentration without green light. The simple model for respiration (FIG. 4a, Eq. 2, solid line) fits the data for spinner velocity without green illumination as a function of azide concentration reasonably well. FIG. 4b shows that exponential decays accurately describe pmf changes for a single cell when green illumination (shaded area) is turned on or off. Finite charging and discharging times are due to membrane capacitance. Decay constants for this cell are 3, 5, 12s charging and 17, 13, 4s discharging. Respiration has been inhibited by 60 mM azide in motility buffer. Angular velocity is averaged in 4 second intervals.

FIG. 5 is a schematic of the operating principle underlying a light-boosted yeast in one embodiment of the invention. In the depicted embodiment, a fraction of the proteorhodopsin is targeted to the mitochondrial membrane of yeast (e.g., by using a Cox4 leader sequence) with the remainder incorporated into other membranes. When illuminated, the PR proton pump extrudes protons (H⁺), helping to maintain a pmf across the inner mitochondrial membrane. This in turn leads to ATP production by the mitochondrial ATP synthase. In addition, the mitochondrial ADP/ATP transporter exchanges cytoplasmic ADP with ATP, boosting cytoplasmic ATP levels, and boosting biomass-to-biofuel conversion yields.

FIG. 6 shows the ability of GFP-tagged proteorhodopsin to reconfigure internal membranes, creating light-collecting structures in yeast. Proteorhodopsin with its native localization signal and fused to a nonmonomeric green-fluorescent protein creates multi-lamellar semicircular membrane structures in yeast. False-color electron micrograph of a single yeast cell shows the synthetically-encoded membrane compartment (3-5 layers thick) surrounding the nucleus.

### DETAILED DESCRIPTION OF THE INVENTION

### I. General

As mentioned above, the numerous embodiments of the invention described herein are variously drawn to recombinant light-harvesting microbes, such as yeast, whose internal energy fluxes can be externally boosted in order to provide additional energy during fermentation and other processes.

### II. Definitions

“Proteorhodopsin” (PR) refers to a light-driven proton pump [O. Beja, et al. (2000), Science 289, 1902-6]. Proteorhodopsins are widely distributed and spectrally tuned to their oceanic environments [O. Beja, et al. (2001), Nature 411, 786-9; G. Sabehi, et al. (2004), Environ Microbiol 6, 903-10; G. Sabehi, et al. (2003), Environ Microbiol 5, 842-9; D. Man-Aharonovich, et al. (2004), Photochem Photobiol Sci 3, 459-62; J. R. de la Torre, et al. (2003), PNAS 100, 12830-5; J. C. Venter, et al. (2004), Science 304, 66-74] and expressed at high levels (˜10⁴ copies per cell) in the ubiquitous gram-negative marine bacterium SAR11 [S. J. Giovannoni, et al. (2005), Nature 438, 82-5]. Proteorhodopsin has sequence homology to sensory rhodopsins and bacteriorhodopsin [O. Beja, et al. (2000), Science 289, 1902-6], but unlike bacteriorhodopsin, PR is active in the heterologous host E. coli [S. Karnik, et al., PNAS 87, 8955-9 (1990); I. P. Hohenfeld, et al. (1999), Febs Lett 442, 198-202; O. Beja, et al. (2000), Science 289, 1902-6]. Bacteriorhodopsin can be modified to be expressed in E. coli as well [I. P. Hohenfeld, A. A. Wegener, M. Engelhard, Febs Letters 442, 198 (1999)].

“Retinal” refers to all-trans retinal, a chromophore required for proton pumping by PR and is synthesized by some proteorhodopsin-containing bacteria [O. Beja, et al. (2000), Science 289, 1902-6; G. Sabehi, et al. (2005), PLoS Biol 3, e273].

“Proton motive force (pmf)” is the electrochemical potential of protons across the membrane. Stoeckenius and Racker demonstrated in the 1970's that the pmf generated by light-driven proton pumping by BR could be used to produce ATP [E. Racker and W. Stoeckenius (1974), J Biol Chem 249, 662-3]. Subsequent studies also using vesicles have revealed how enzymes control membrane formation [D. W. Deamer and D. E. Boatman (1980), J Cell Biol 84, 461-7] and characterized numerous biological energy conversion complexes, including ATPases [S. M. Goldin (1977), J Biol Chem 252, 5630-42] and photosystem II [B. Andersson, et al. (1977), FEBS Lett 77, 141-5], some of which are now being combined with synthetic components to construct artificial photosynthetic membrane systems capable of producing ATP [G. Steinberg-Yfrach, et al. (1998), Nature 392, 479-82].

Bacteria use the pmf to drive chemiosmotic reactions and power the rotary flagellar motor (reviewed in [R. M. Macnab, in Escherichia coli and Salmonella: Cellular and Molecular Biology, ASM Press (2005); S. C. Schuster and S. Khan (1994), Annu Rev Biophys Biomol Struct 23, 509-39]). Under aerobic conditions, the requisite electrochemical gradient is maintained by oxidative phosphorylation. As many as 1000 protons are required per turn of the E. coli flagellar motor, and the motor's speed is proportional to the pmf over a wide range of speeds [C. V. Gabel and H. C. Berg (2003), PNAS 100, 8748-51; D. C. Fung and H. C. Berg (1995), Nature 375, 809-12].

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” in the context of a nucleic acid or protein denotes that the nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993).

The phrase “a nucleic acid sequence encoding” refers to a nucleic acid that contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “Table #” when used in the specification includes all sub-tables of the Table referred to unless otherwise indicated.

### III. Recombinant Light-Harvesting Microbes

The recombinant light-harvesting microbes described herein may be derived from yeast, such as S. cerevesiae, or other organisms depending on the microbe's desired properties. In some instances, microscopic entities with properties similar to the exemplary recombinant light-harvesting microbes described herein could be generated using in vitro preparations of cell membranes or artificial lipid membranes as a starting material.

S. cerevisiae has traditionally been used in ethanol synthesis. S. cerevisiae has several advantages compared to other microbes with respect to conversion of biomass to ethanol: (1) S. cerevisiae displays high tolerance to ethanol and to the toxic by-products present in lignocellulose hydrolysates; (2) S. cerevisiae genetics are well-known; and (3) S. cerevisiae is publicly considered a safe microorganism.

The composition of biomass is approximately 15-40% cellulose, 30-40% hemicellulose and pectin, and 20% lignin. Much progress has been made towards developing strains of S. cerevisiae that can use some of these substances or the products of their degradation for the synthesis of ethanol. For example, strains RWB 218 and 424A(LNH-ST) have been engineered to ferment xylose. Xylose is the product of the degradation of the hemicellulose xylan, which is the second most abundant carbohydrate in nature. Yeast strains have been engineered with the ability to degrade cellulosic products and even amorphous cellulose to ethanol.

The cellular energetics of aerobically and anaerobically growing microbes are quite different. In aerobic conditions, the cytochromes in the respiratory chain extrude protons, creating a pmf. These protons then return into the cell (or mitochondrion) via the rotary ATPase, converting ADP+Pi into ATP. In anaerobic growth (fermentation), ATP is instead generated by glycolysis, yielding two ATPs per glucose. Unlike cells in aerobic conditions, cells in anaerobic conditions are likely to be severely energy constrained. One of many consequences of their energy limitation is that carbon sources in the cell's environment are incompletely utilized: only 10% of the available carbon sources are assimilated during fermentation due to poor energy yields.

One consequence of cellular energy limitation during fermentation is poor xylan degradation. Xylose, a degradation product of xylan, can be fermented by certain strains of S. cerevisiae, but only inefficiently. Another consequence of cellular energy limitation during fermentation is poor sugar-to-fuel conversion efficiency. When S. cerevisiae cells are grown in a chemostat with glucose as the only limiting substrate, energy limits biofuel production instead of carbon.

The invention described herein provides, in one embodiment, a photosynthetic yeast based on proteorhodopsin (PR) expression. Proteorhodopsin is a light powered proton pump used by some ocean bacteria to scavenge light energy. By illuminating single yeast cells expressing PR, controlled amounts of energy can be obtained by these cells and used to achieve useful results. This extra energy in the form of light-induced ATP will boost biofuel production without diverting energy from other cellular energy consuming reactions.

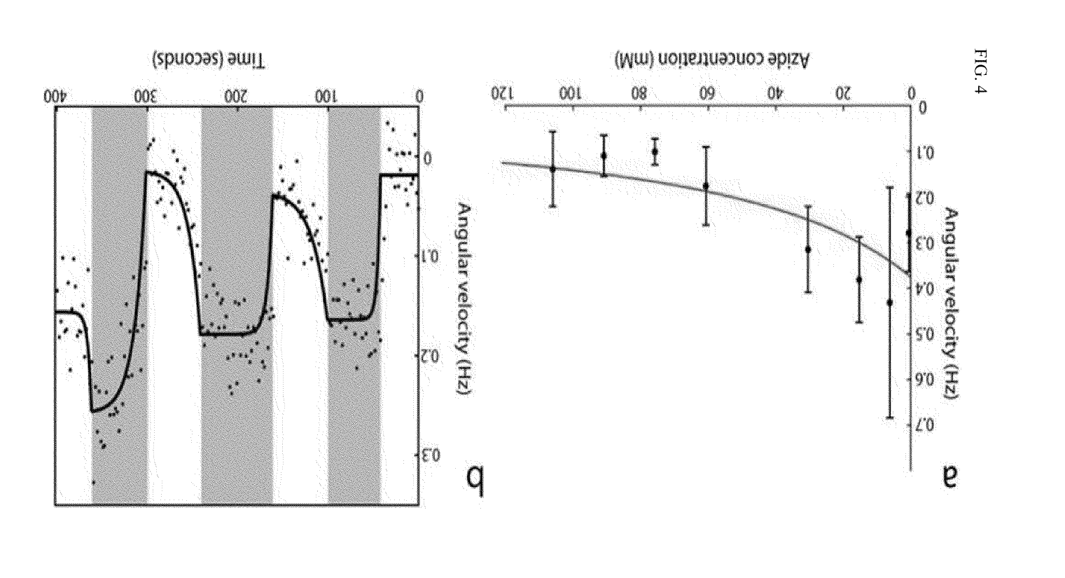

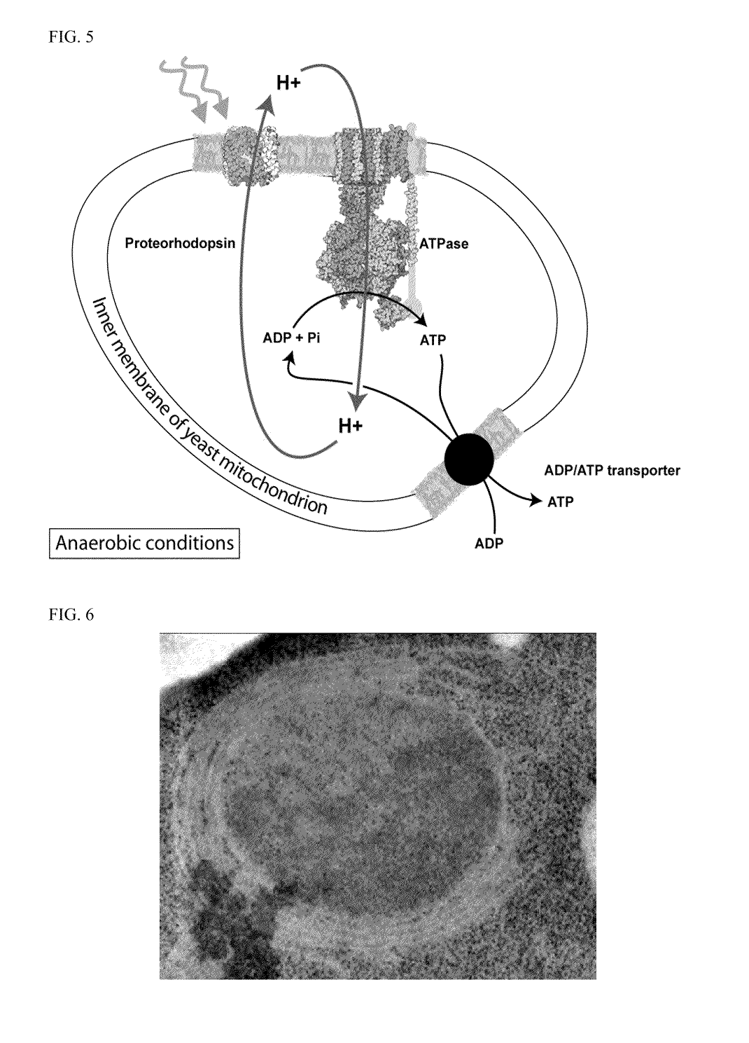

The presumptive operating principle is shown schematically in FIG. 5. Unlike the E. coli work referenced herein, yeast cells can be obtained wherein the light-powered proton pumps, e.g., proteorhodopsin, are present in more than membrane. For example, two distinct PR derivatives can be expressed in a single yeast cell. A fraction of the expressed PR is targeted to their mitochondria by the Cox4 leader sequence, while the remainder is incorporated into the plasma membrane. When illuminated, the PR proton pump extrudes protons (H+), helping to maintain a pmf across the inner mitochondrial membrane. This in turn leads to ATP production by the mitochondrial ATPase. Finally, the mitochondrial ADP/ATP transporter exchanges cytoplasmic ADP with ATP, boosting cytoplasmic ATP levels, and thereby boosting biomass-to-biofuel conversion yields. Experiments characterizing light-harvesting yeast cells are presented in the Examples, below.

A. Enhanced Drug Resistance and Biofuel Production Using Recombinant Light-Powered Cells

As discussed above, proton-motive forces constitute a central energy store of the cell. Once generated, a proton motive force (pmf) can be used by the cell to perform a multitude of reactions of interest to medicine, energy, industry, and defense. For example, the pmf generated by light-capture can be used directly to power molecular machines such as the flagellar motor, yielding cells that swim when illuminated.

The energy stored in the pmf can also be used to synthesize energy-rich molecules such as adenosine tri-phosphate (ATP). The cell can then use the energy-rich molecules to convert biomass into biofuels. In another embodiment, ATP generated by light-capture can be used to power ABC superfamily drug transporters, yielding cells that resist certain drugs if and only if illuminated with particular colors of light, some of which may penetrate deep inside tissues.

The pmf generated by light-capture can also be used by the cell to assist with the transport of metabolites across membranes such as the plasma membrane, thereby decreasing the amount of energy the cell must divert from anabolism to take-up (or extrude) metabolites from (or into) the medium.

Depending on the particular combinations of chosen light-powered proton pumps and cell membranes in which those pumps are expressed, organisms can be produced with a variety of cell physiologies that differ from those found naturally. For example, by targeting PR to both the inner mitochondrial membrane as well as the plasma membrane, a strain of yeast can be synthesized with increased biomass to ethanol conversion efficiency. Similarly, a strain of yeast can be synthesized that features increased growth under oxygen-poor conditions, or that exhibits drug resistance in a light-dependent manner, or both.

Proper function and control of the organisms described above, e.g., the recombinant yeast, requires at least (a) efficient targeting of light powered proton pumps to particular cell membranes and (b) simultaneous modulation of the cell's natural pmf generating and modulating reactions.

Proper function and control of the organisms described above require the use of synthetic biological approaches to create the cellular ultrastructures that enhance the function of the light-powered proton pumps. In some embodiments, the cellular ultrastructures are created by expressing certain derivatives of the light powered proton pump proteorhodopsin in the organism. For example, under certain conditions, expression of the light powered proton pump proteorhodopsin in S. cerevisiae results in the spontaneous emergence of layered membrane stacks reminiscent of chloroplasts in plants. Another requirement is that a significant fraction of the encoded light-powered proton pump is properly folded and contains a chromophore. In this regard, the invention provides a method for modifying the codon usage to reduce the translation rate. This reduction in translation rate greatly increases the fraction of functional enzyme in the membrane.

B. Proteorhodopsin as an Energy Source

As discussed above, the entities of the present invention require an energy source, a means for capturing the energy, and a means for storing and then converting the captured energy into “work,” i.e., into the performance of tasks specified by the controller of the entity or entities. The Examples provided herein describe entities which use heterologously-expressed proteorhodopsin as a means for capturing light energy and converting light energy into a proton motive force. The proton motive force may then be used to drive any number of processes which require energy, e.g., mechanical movement or chemical synthesis. One skilled in the art will recognize that, depending on the functionality desired of the recombinant light-harvesting microbe, different heterologous proteins for converting and/or storing energy may be employed for different tasks, or one source of energy may be used to modulate a task which relies primarily on a second source. Also, depending on the organism from which the recombinant light-harvesting microbe is derived, different sources of energy, e.g., pmf, may be more easily incorporated and more easily manipulated. For example, bacteriorhodopsin may used in lieu of proteorhodopsin. Another useful pump is xanthorhodopsin, e.g., the proton-pumping retinal protein/carotenoid complex in the eubacterium Salinibacter ruber [Balshov et al., Science 309:2061 (2005)]. Proteorhodopsins with different absorption spectrums are known in the art (see, e.g., U.S. patent application Ser. No. 09/847,513, published as US20030104375). One skilled in the art will recognize that certain modifications to these proteins may be necessary in order to achieve optimal functionality in their desired hosts.

A heterologous proteorhodopsin or other source of pmf may be introduced into a bacterium or other microorganism using genetic engineering techniques which are well-known to those of skilled in the art. For example, bacteria may be transformed with one or more vectors such as plasmids or viruses which encode the genes encoding proteorhodopsin. The Examples provided herein utilize plasmid vectors, but microbial strains in which the promoters and structural genes encoding proteorhodopsin (or other source of cellular pmf) are incorporated intrachromosomally may also be used. Methods for inserting genes into microbial chromosomes using homologous recombination, site-specific recombination, transposition, etc., are also well-known in the art.

If heterologous proteorhodopsin is used as a source of cellular pmf, the addition of the chromophore all-trans-retinal (typically referred to herein as “retinal”) may be necessary in order for proton-pumping by proteorhodopsin to occur. The amount of all-trans retinal needed in a recombinant light-harvesting microbe which comprises heterologous proteorhodopsin may vary depending on the desired functionality of the recombinant light-harvesting microbe. Retinal concentrations may be varied, for example, to provide a recombinant light-harvesting microbe with varying degrees of sensitivity, thus providing another means of controlling a parameter of the recombinant light-harvesting microbes behavior, e.g., maximum speed or maximum flagellar velocity. In the Examples provided (see, e.g., Example 1), ethanolic all-trans-retinal is provided exogenously at concentrations of 10 μM. This concentration may be varied several-fold, at least, as desired by the controller of the recombinant light-harvesting microbe.

Recombinant light-harvesting microbes expressing heterologous proteorhodopsin may be engineered to express retinal autonomously by providing the parent bacterium with the genes necessary for all-trans-retinal synthesis. For example, a plasmid may be employed such as pACCAR16ΔcrtX plasmid [N. Misawa, J. Bacteriol., 177(22):6575-84 (1995); Y. Nishida, Appl. Env. Microbio., 71(8):4286-96 (2005)] (which encodes a carotenogenic gene cluster) augmented with a gene encoding apocarotenoid-15,15′-oxygenase (ACO) [D. Kloer, Science, 308(5719):267-9 (2005)]. Such a plasmid contains all the genes needed for E. coli to synthesize retinal. Protoeorhodopsin-expressing bacteria containing this plasmid would produce recombinant light-harvesting microbes with functional proteorhodopsin. Genome-less recombinant light-harvesting microbes such as those described in Example 4 and elsewhere herein could similarly be prepared by providing a parent bacterium with these genes.

The invention described herein provides recombinant light-harvesting microbes with increased sensitivity to environmental factors which regulate cellular pmf, e.g., factors such as light. This increased sensitivity is necessary if the environmental factor (e.g., light) is used as a means for controlling the behavior of the recombinant light-harvesting microbe. In the case of light-controlled recombinant light-harvesting microbes comprising heterologous proteorhodopsin, it is demonstrated herein that reducing the natural source of cellular pmf allows recombinant light-harvesting microbes to be controlled with increased sensitivity, such that the rates of proton-powered motors and other proteins is proportional to light intensity over a wide range.

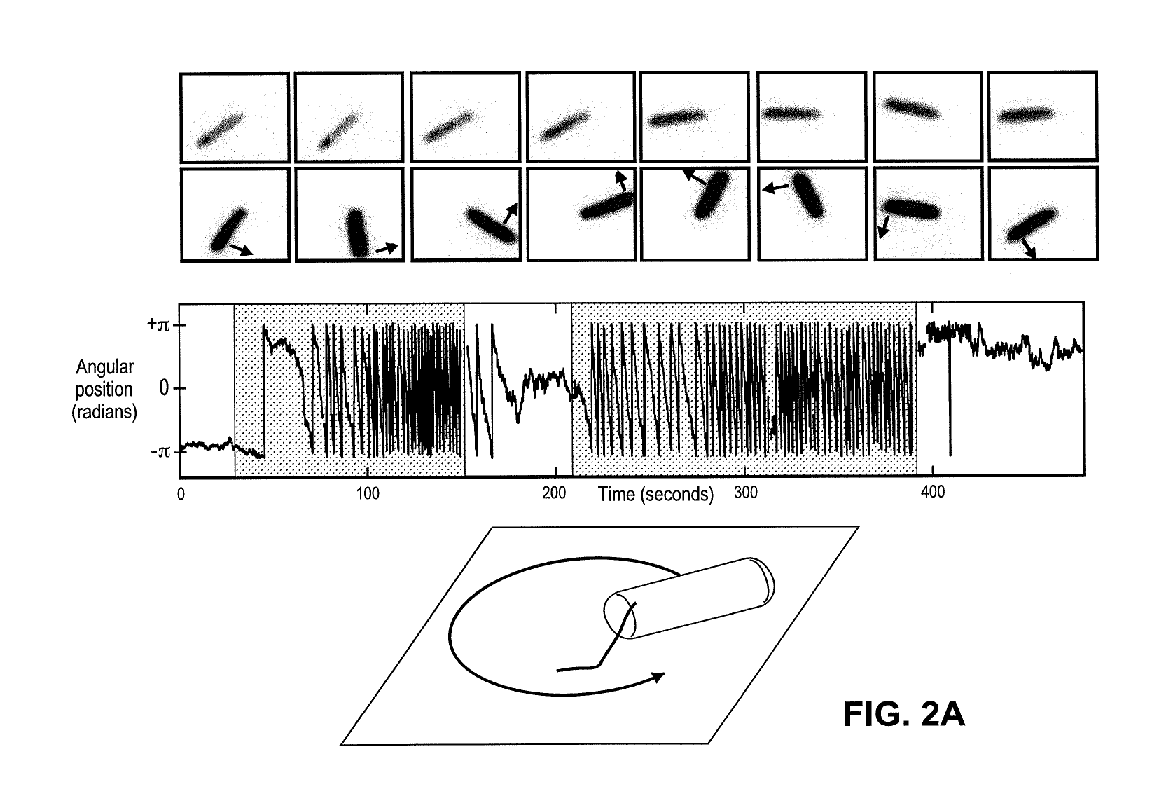

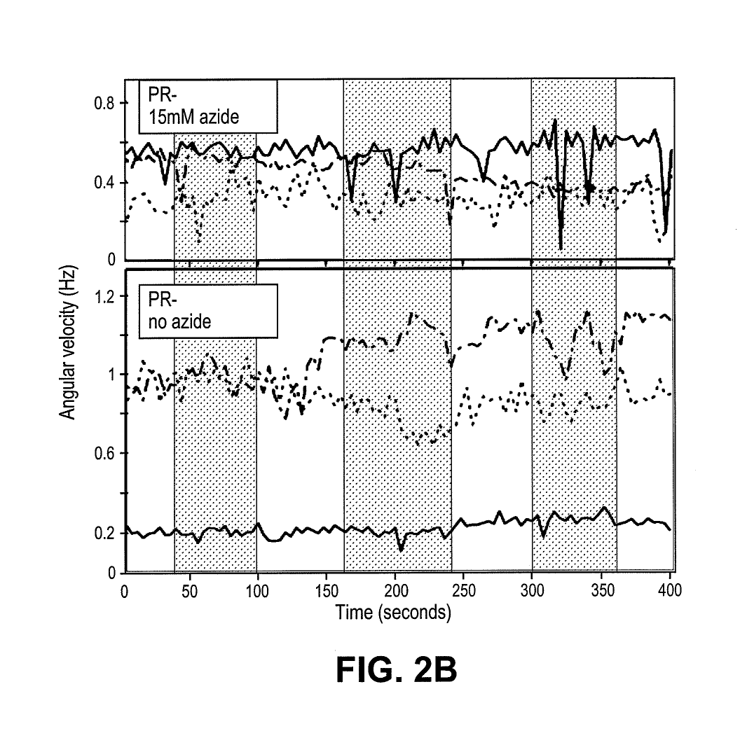

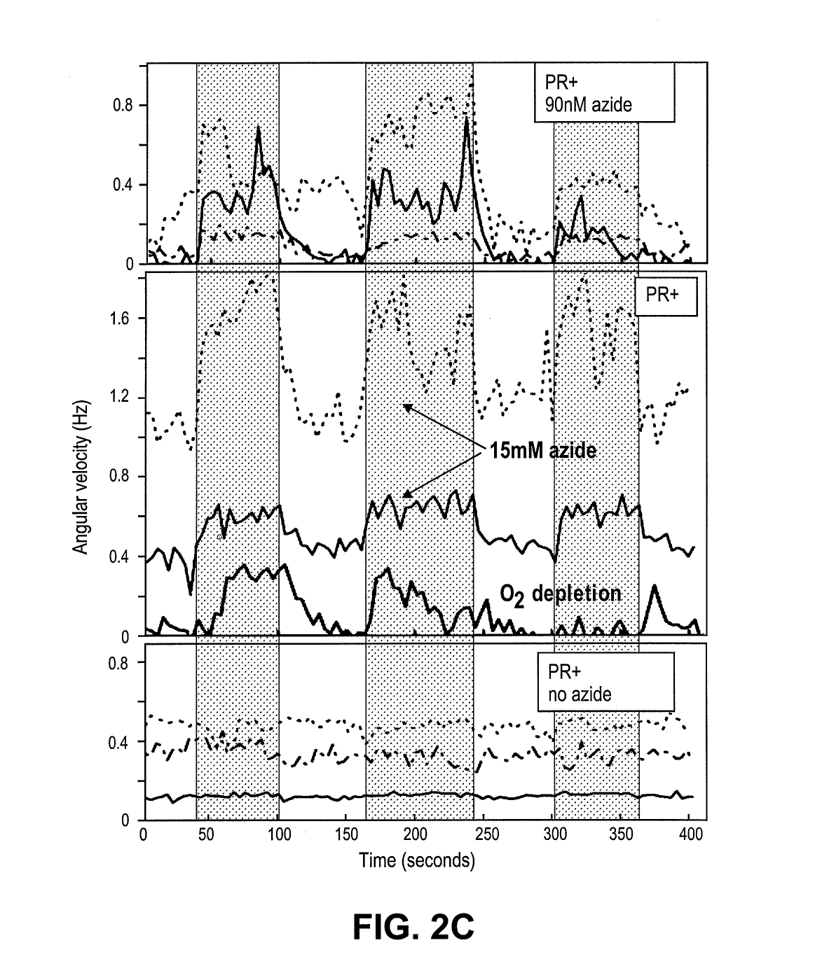

The Examples provided herein show a number of approaches which, independently or cumulatively, reduce a recombinant light-harvesting microbe's natural source of cellular pmf. The recombinant light-harvesting microbe is then dependent on an “artificial” source of pmf (e.g., heterologous proteorhodopsin), thereby allowing the recombinant light-harvesting microbe's controller to “turn on” the recombinant light-harvesting microbe and manipulate its pmf-dependent actions at will. As shown in the Examples, one effective means of reducing natural sources of pmf in a bacterial cell is to introduce an inhibitor of cellular respiration, such as sodium azide. For a recombinant light-harvesting microbe derived from a gram-negative bacteria such as E. coli, concentrations of azide in the ranges of approximately 1 mM to 150 mM are preferred, with higher concentrations causing greater inhibition and, therefore, greater sensitivity and responsiveness to light, as shown in Example 2 (see, e.g., FIG. 2 and FIG. 4).

Another method demonstrated herein of inhibiting respiration and increasing recombinant light-harvesting microbe controllability is to deplete the supply of oxygen available to the recombinant light-harvesting microbe. This method may be used independently or in conjunction with the azide inhibition method described above. Various methods of depriving cell cultures of oxygen are known in the art. The exemplary protocol provided in Example 2 utilizes nitrogen and a sealed chamber.

Regardless of the method used to modulate (in this case, inhibit) a light-powered recombinant light-harvesting microbe's natural source of pmf, the natural source of pmf should be modulated significantly to achieve meaningful control over pmf-driven activities. Specifically, cellular respiration should be inhibited by at least approximately 10%, and more preferably by 20% to approximately 100%, relative to respiration in the absence of an inhibitor or oxygen-depleting scheme. When inhibition of a cell's natural source of pmf approaches 100%, nearly complete control of heterologously-produced cellular pmf may be achieved.

C. Light Sources

In some embodiments, the controllable microbiologically-derived entities of the invention are controlled, at least in part, by exposure to light. As described above, recombinant light-harvesting microbes which comprise heterologous proteorhodopsin may be engineered according to the invention to respond to manipulated light. Proteorhodopsin and proteorhodopsin-like molecules are typically activated to generate cellular pmf by particular light frequencies. For example, the proteorhodopsin used in the exemplary embodiments herein has an absorbance maximum of approximately 525 nm (corresponding to “green” light). Thus, recombinant light-harvesting microbes with the particular proteorhodopsin described in the Examples can be selectively maximally activated by exposure to a light source tuned to that frequency, whereas other recombinant light-harvesting microbes with different rhodopsins may remain “off” or activated to perform at a reduced level.

Any type of light source can be used within the scope of the invention, provided that the light source delivers light at a frequency and intensity which is absorbed by the recombinant light-harvesting microbe's heterologous light-absorbing rhodopsin pigment. The light emanating from the source can be manipulated by the controller in any manner, including changing the frequency of light emitted, pulsing the light on and off, switching back and forth between frequencies, changing intensity, etc. Manipulating a recombinant light-harvesting microbe's exposure to light thus provides a means of sending signals to a recombinant light-harvesting microbe. The signals can be used to specify discrete tasks to be performed by the recombinant light-harvesting microbe, particularly when combined with the variations in proteorhodopsins, retinal concentrations, concentrations of inhibitors of natural respiration, and the presence of chemicals for modulating cellular pmf or other cellular circuits, as described herein.

D. Movement of Recombinant Light-Harvesting Microbes

In embodiments of the invention where a recombinant light-harvesting microbe comprises a heterologous source of pmf, the pmf so generated can be used for any number of purposes. In some embodiments, for example, the pmf can be used to drive subcellular “machines” or molecular motors. A quintessential example of such a motor is the bacterial flagellum, but any proton-driven motor will suffice for the purposes of the invention.

The exemplary embodiments described herein utilize the flagellum of E. coli, but other pmf-powered flagella may be used. For certain purposes, e.g., creating genome-less recombinant light-harvesting microbes with one or more flagella, overexpression of one or more flagellar proteins in the parent cell may be desirable (see, e.g., Example 4). Alternatively, or additionally, recombinant light-harvesting microbes may comprise mutations in a chemotaxis or flagellar synthesis pathway which alter the properties of the flagellum, cellular motility, or both. For example, a knockout mutation in the bacterial gene cheY, one the genes involved in the control of tumbling during chemotaxis, decouples control of the direction of flagellar rotation from the cells natural chemotactic signal processing pathway. A recombinant light-harvesting microbe comprising such a mutation or otherwise incapacitated with respect to the activity of the CheY protein will be smooth-swimming, i.e., it will not tumble as bacteria normally do during chemotaxis.

Depending on the application for which the recombinant light-harvesting microbe is designated, other aspects of the flagellum may be varied. For example, the number of flagella may be varied and/or the relative position of the flagellum or flagella with respect to the cell poles. In some embodiments, the recombinant light-harvesting microbe may be anchored to a substrate and the turbulent force generated by the flagellum may be used to disturb the liquid medium in which the recombinant light-harvesting microbe is positioned. Such turbulence may be used to distribute solutes throughout a solution, or to create a current for moving larger insoluble materials. In other embodiments, the flagellum may be anchored to a substrate, allowing the recombinant light-harvesting microbe to be turned and positioned at the controller's discretion. Various means for temporarily or permanently anchoring flagella or bacteria to a substrate may be employed by one of skill in the art, including chemical cross-linking.

The action (i.e., rotation) of the flagellum of the recombinant light-harvesting microbe can be controlled by various means, including light, as described herein. For example, in addition to light, various chemical agents may also be used to direct or influence the movement of the recombinant light-harvesting microbe. As shown in Example 1, herein, a protonophore such as carbonyl cyanide 3-chlorophenylhydrazone which decouples cellular pmf may be used to stop flagellar rotation.

E. Applications of Recombinant Light-Harvesting Microbes

In various embodiments, the invention provides methods of exploiting recombinant light-harvesting microbes to achieve defined goals. As discussed above, the exceedingly small size of recombinant light-harvesting microbes makes them ideal for effecting controlled, microscopic changes to their environment. In the simplest implementation, the change to the environment is simply the presence of one or more recombinant light-harvesting microbes at a particular location. In other implementations, the recombinant light-harvesting microbes may perform a specified task at the desired location, such as the deposition of a recombinant light-harvesting microbe-synthesized protein, nucleic acid, or other chemical. Alternatively, or in conjunction with such a deposition, the recombinant light-harvesting microbes may enzymatically alter a surface, removing specific molecules at a location, or etching a coated surface continuously as they travel across it.

Given their responsiveness to light signals, recombinant light-harvesting microbes comprising heterologous rhodopsins are particularly useful for lithographic applications. For example, a very simple biophotolithographic application using recombinant light-harvesting microbes comprising heterologous proteorhodopsin would involve exposing a population of recombinant light-harvesting microbes on a two-dimensional surface to a pattern of light. The pattern would consist of continuously displayed red light and green light (e.g., light at the absorption maximum of proteorhodopsin). Exposure to green light would signal recombinant light-harvesting microbes to move, i.e., the green light would generate cellular pmf which would allow flagellar rotation to commence. The recombinant light-harvesting microbes exposed to green light would continue moving until they entered an area which was not exposed to green light, e.g., an area exposed to red light. In the absence of green light, the recombinant light-harvesting microbe's cellular pmf would soon be depleted and the recombinant light-harvesting microbe will cease moving. In this way, a pattern of recombinant light-harvesting microbe cells on a surface can be generated which corresponds to the regions of the surface which were not exposed to the movement-activating green light. The patterns of light in this simple example could be generated using a lithographic mask, or by using light sources which emit focused light at different frequencies, or any other means capable of creating a distinct pattern of light on a surface.

In combination with the other recombinant light-harvesting microbe signalling possiblities discussed herein, the biophotolithography methods of the invention may be used for preparing microarrays such as those used in clinical diagnosis and biological research. The methods may also be used for creating “chips” or any other surface where microscopic patterning is desired.

F. Formation of Membrane Shelves in Recombinant Light-Harvesting Microbes

In some embodiments, the invention provides recombinant light-harvesting microbes that exhibit a membrane structure having a plurality of layers of a membrane. It is proposed that these recombinant microbes having a plurality of membrane layers will exhibit an increased ability to pump protons in response to illumination, due to the increased capture cross-section for photons and effective light collection that the membrane layers provide.

The principle behind the utility of membrane folding in increasing proton pumping in recombinant light-harvesting microbes is illustrated by, for example, photoreceptor cells in the human eye or chloroplasts in plant cells. In photoreceptor cells, for example, the plasma membrane is folded back on itself numerous times. These “membrane stacks” or “membrane shelves” increase the surface area of the cell membrane and, therefore, the capture cross-section for photons. Without this multilayered membrane structure, the human eye would be hundreds of times less sensitive to light.

In one embodiment of the present invention, a synthetic light-harvesting structure is created in a microbe by expressing a modified form of the proteorhodopsin light-powered proton pump. Expression of a modified PR construct results in the formation of multi-lamellar semicircular membrane shelves which are highly enriched for PR and reminiscent of plant chloroplasts. By influencing membrane structure and creating membrane shelves, the modified PR construct transforms certain organelles of the microbe in precisely the right way to theoretically maximize the microbe's proton-pumping efficiency.

It is proposed that the formation of multiple layers of membrane in recombinant light-harvesting microbes is the result of protein dimerization or oligomerization. Therefore, it is contemplated that a gene encoding a proteorhodopsin light-powered proton pump may be modified in any way so as to promote dimerization or oligomerization. In some embodiments, the gene may encode a fusion protein of a proteorhodopsin polypeptide fused to a non-proteorhodopsin polypeptide, domain, or motif that promotes dimerization or oligomerization. In some embodiments, the gene may encode a fusion protein of a proteorhodopsin polypeptide fused to a nonmonomeric fluorescent protein tag. In some embodiments, the nonmonomeric fluorescent protein tag comprises a nonmonomeric green fluorescent protein (GFP) such as an Aequorea GFP, a nonmonomeric red fluorescent protein (RFP) such as a Discosoma RFP (DS-Red), or any nonmonomeric fluorescent protein related to GFP or DS-Red. In some embodiments, the gene may encode a fusion protein of a proteorhodopsin polypeptide fused to a small-molecule dimerizer domain. One skilled in the art will recognize other suitable modifications, additions, and/or deletions that can be made to a proteorhodopsin construct in order to promote dimerization or oligomerization. Methods of making these modifications, additions, and/or deletions are well-known in the art.

### IV. Examples

The following Examples are provided to illustrate various aspects of the invention described herein. As such, they are not necessarily the best or most preferred embodiments of the invention.

### Example 1

Cell culture. We expressed the SAR86 γ-proteobacterial PR-variant (Geneart, Inc) in E. coli cells (RP437 DE3 ΔcheY Cm^{R}) using a T7 based expression system (pET200, Kan^{R}, Invitrogen). Cells were grown in T broth (1% Tryptone, 0.5% NaCl) supplemented with kanamycin (25 μg/ml). In mid-log phase, PR expression was induced with 1 mM IPTG and the medium was supplemented with 10 μM ethanolic all-trans-retinal. Cells were collected in late log phase by gentle centrifugation (4500×g, 5 min) and carefully resuspended in motility medium (1 mM PBS (Ambion), 0.1 mM EDTA, pH 7.4). PR⁺ denotes cells expressing proteorhodopsin, and PR⁻ denotes cells (RP437 DE3 ΔcheY Cm^{R}) without the PR plasmid. PR⁻ cells, unless otherwise noted, were also induced with IPTG, supplemented with all-trans-retinal and grown in T broth with chloramphenicol (25 μg/ml). Unless otherwise noted, all experiments were done in glucose free motility medium and at room temperature.

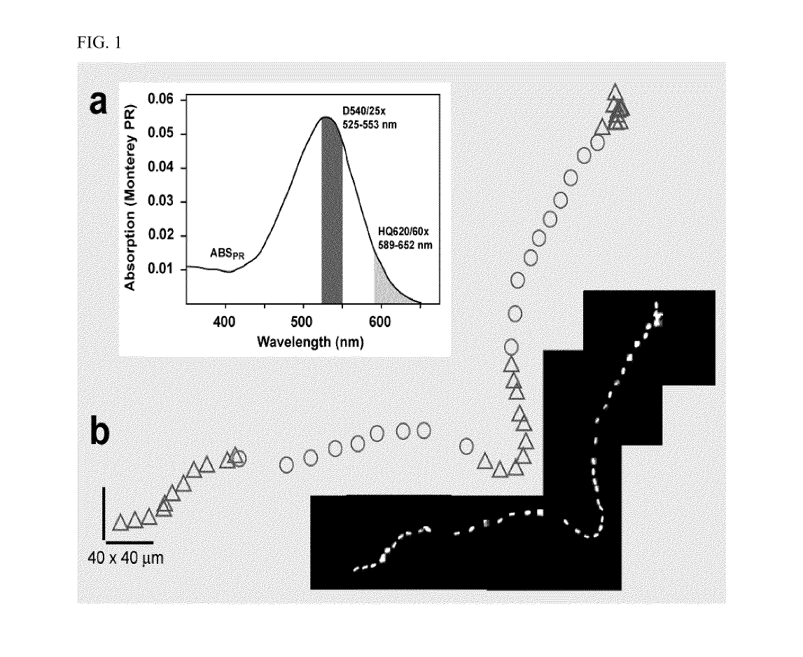

Instrumentation. Power density values for “green light” refer to the power density passed by a D540/25x filter (Chroma Inc.) and originating at a 175 W Xenon bulb (Lambda light source, Sutter Instruments), or, for “red light,” to the power density passed by a HQ620/60x filter and originating at a 100 W Quartz Halogen bulb (Nikon). To visualize the cells, the sample chamber was continuously illuminated with faint red light (0.09 mW/cm²) at the tail of PR's absorption spectrum (4) (FIG. 1A). We periodically illuminated the sample chamber with bright green light (160 mW/cm²) coinciding with the maximum of PR's absorption spectrum, ˜525 nm (4). Cells were imaged at a frame rate of 5 Hz using an Andor iXon camera mounted to a Nikon TE2000 microscope. The Nikon microscope was modified for dark-field work by attaching a Zeiss 1.2-1.4 NA oil immersion dark-field condenser to the Nikon condenser turret using a custom adapter. Custom software written in C++ was used to control the Andor camera, and Matlab was used to process the images. All errors and error bars represent standard errors of the mean.

Cell viability experiments. PR⁺ and PR⁻ cells were grown in LB+Kanamycin (PR⁺) or LB+Chloramphenicol (PR⁻) to an OD₆₀₀ of 0.25-0.3, induced with 1 mM IPTG and supplemented with 10 μM all-trans-retinal (if retinal+), grown to an OD₆₀₀ of 0.5-0.6, spun down (4500×g, 5 min), resuspended in motility buffer (1 mM PBS, 0.1 mM EDTA) to an OD₆₀₀ of 0.1-0.2. One to two ml of each culture was placed in a clear plastic culture tube, sodium azide was added to 30 mM, and tubes were placed outside in the sun. Samples were taken before addition of azide (0 min) and after 30 minutes of exposure to sunlight. 50 μl of each sample was plated at dilution of 1/10⁴ (0 minutes) or 1/10³ (30 minutes) onto LB with the appropriate antibiotic. Cell density at 0 minutes (prior to the addition of azide) was ˜3×10⁶ colonies in 50 μl, a density that matches well with the measured OD₆₀₀ of 0.1-0.2. Colonies were counted manually following overnight incubation. The one-sided Mann-Whitney U test (31) was used to test the null hypothesis that the ability to pump protons does not increase cell survival.

Swimming PR⁺ E. coli were tracked in two dimensions using dark-field microscopy, periodically illuminating the cells with green light at PR's absorption maximum. No detectable increase in cell swimming velocity was observed upon illumination with green light. However, energy-depleting the cells using the respiratory poison sodium azide converted the cells into controllable entities, responsive to green light. Sodium azide has multiple cellular effects (22, 23) but primarily inhibits cytochrome oxidase and, as a result, proton extrusion by the respiratory chain, which ordinarily stops the flagellar motor in bacteria like E. coli (18).

With respiration inhibited by azide, PR⁺ cells responded to green light. PR⁺ cells in 30 mM azide swam slowly in red illumination but showed a marked velocity increase with green illumination (FIG. 1B). Upon removal of the green light, the bacteria slowed to their previous velocity. To increase the accuracy of the flagellar rotation measurements, a tethered cell geometry was employed (FIG. 2A) which permitted the extended observation of the same bacterium in different illumination conditions. PR⁺ cells were allowed to stick to the coverslip via a flagellum, and the angular rotation rate of cells was monitored (FIG. 2A). A typical tethered cell rotated at a mean rate of 0.2-1 Hz, depending on its length and the position of the stuck flagellum along its body. Deleting the cheY gene (24, 25) yields smooth-swimming mutants whose flagellar motors do not reverse.

In the absence of azide, green light did not affect the cell's rotation rate (FIG. 2B). However, as respiration was inhibited by adding azide, the cells again became light-responsive and PR⁺ cells sped up upon illumination with green light (FIGS. 2A and C). The PR⁺ cells converted light energy into an electrochemical potential used to do mechanical work. PR⁺ cells stopped moving or slowed considerably when the green illumination light was removed. For example, at low concentrations of azide (5-15 mM), angular velocity dropped by one fourth when the green light was removed. At higher azide concentrations (80-110 mM), angular velocity dropped further (50-60%) because more of the cells' cytochromes were bound by azide (26). Thus, the sensitivity of cells to light is controllable by modulating azide concentration.

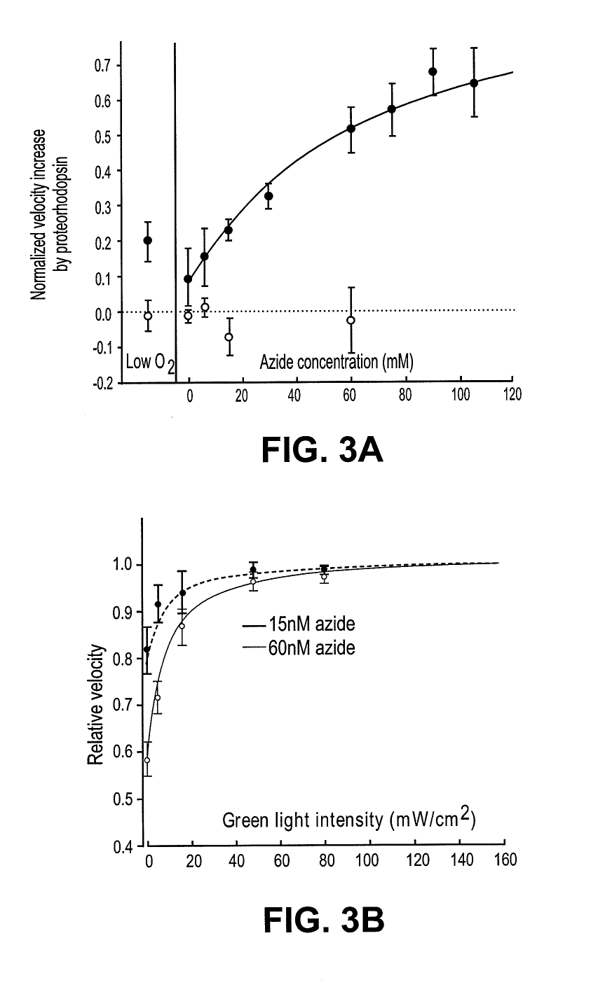

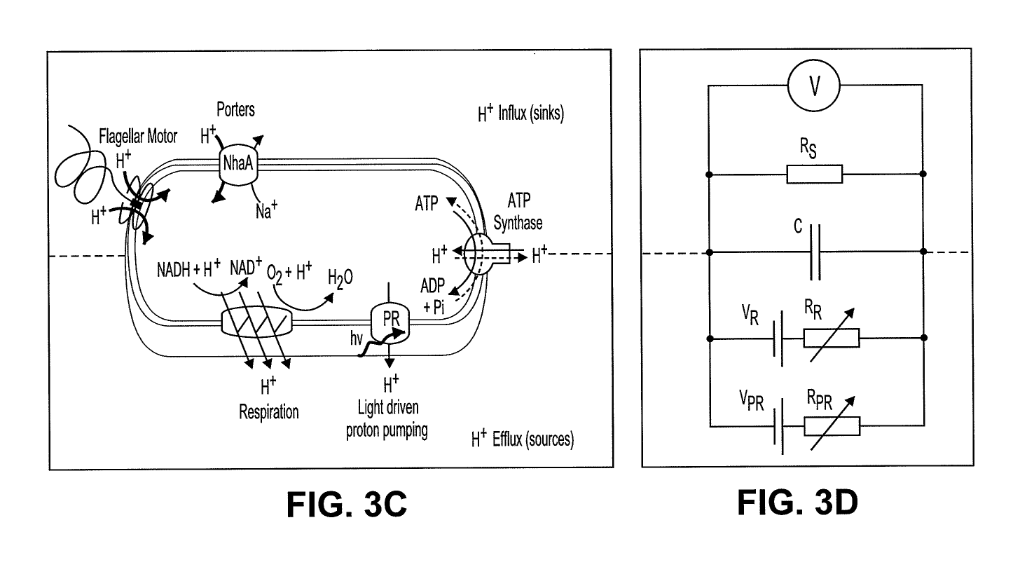

Light can replace respiration as an energy source to varying extents. Increasing the azide concentration from 0 to 110 mM caused cellular pmf levels, and therefore average cellular angular velocity, to decrease (FIG. 4). The pmf dropped to 50% of its original value at an azide concentration of 55 mM. When the cells were illuminated with the intense green light, their rotation rate was restored to the speed of cells with an unimpaired respiratory system. As proton extrusion by the respiratory system dropped with increasing azide, PR provided an ever larger fraction of the proton efflux needed to maintain the cellular pmf at the original, no-azide value (FIG. 3A). At the highest azide concentration studied, the average cell velocity increased 70% upon green light illumination. Cells lacking PR exhibited no increasing angular velocity in green light, indicating that PR expression is required.

The intensity of the green light illumination can be varied and used to control proportional changes in rate. The rotation rate was clearly stimulated even at the lowest light intensity studied (˜5 mW/cm²) (FIG. 3B). The rate increased rapidly with intensity up to 10 mW/cm² (15 mM azide) or 20 mW/cm² (60 mM azide) (FIG. 3B), where the effect saturated. Above 50 mW/cm², there was no detectable benefit of increased illumination.

Removal of oxygen from the cell culture also leads to light-responsive bacteria. O₂ levels were reduced by gently bubbling nitrogen through the cell culture for 15 minutes. A nitrogen-filled glove-box was then used to prepare sealed imaging chambers containing 2 μl of cells. As was observed upon azide addition, PR⁺ cells became light-responsive upon oxygen depletion. Illumination increased the cells' angular velocity by 45±25% (FIG. 2c, p<0.005, one-sided t-test). Addition of the protonophore carbonyl cyanide 3-chlorophenylhydrazone (CCCP, 100 μM) to low oxygen cultures eliminated all bacterial motility, and movement was not restored with 50 seconds of green illumination (N=300), demonstrating that cells unable to maintain a pmf cannot be revived.

### Example 2

FIG. 3C shows a highly simplified model of E. coli membrane fluxes. The PR+ cells described in this Example have multiple proton pumps that can contribute to the pmf (28), including PR, the respiratory chain, and the ATPase (FIG. 3C). Pmf is consumed by the flagellar motor and numerous transporters. In addition, the bacterial membrane has a basal permeability to protons (29). The model shown in FIG. 3D has been paramaterized by fitting it to the data shown in FIGS. 3A and 3B, and in FIG. 4. This model describes in vivo time-dependent dynamics between light-driven proton pumping and respiration. Despite the model's simplicity, it suggests why no effect of PR on growth rates has been reported. The model indicates that the maximum potential PR can generate using the free energy from photon absorption (V_{PR}) is similar to the potential generated by E. coli respiration. Thus, according to this model, PR cannot pump protons in E. coli grown at neutral pH in rich or minimal media, or in E. coli respiring aerobically using endogenous energy stores. Only when the pmf falls below the maximum potential (V_{PR}) during respiratory stress does PR begin to pump, and the proton flux through PR increases as the pmf falls. With sufficiently bright illumination (K_{M}≈60 mW/cm²), PR is able to maintain E. coli cellular pmf near this maximum potential (V_{PR}≈0.2V).

### Example 3

In addition to powering the flagellar motor, this Example shows that PR can pump sufficient protons to increase cell viability. Cell cultures were plated following their exposure to 30 mM azide for 30 min in sunlight. The cells lacking retinal were most sensitive to azide. No colonies were recovered after plating the azide exposed cells. The cells lacking PR but having retinal were slightly more azide resistant (1% percent of cells survived; Table 1). The cells with both PR and retinal were significantly more azide resistant than in all three other conditions (11% of cells survived, p<0.005, Mann-Whitney U test). Thus, consistent with the motility studies and the pmf model, PR is able to sustain cellular pmf at a level that increases viability.

TABLE 1
Effect of sunlight on cell viability following azide exposure

| | | Null hypothesis: the ability |
|---|---|---|
| | Percentage of cells surviving | to pump protons does not |
| Case | azide and light exposure | increase cell survival |
| PR+RET+ | 3.4, 9.3, 2.2, 3.7, 4.6, 27, 0.8, 39 | NA |
| PR+RET− | 0, 0, 0, 0 | p < 0.001 |
| PR−RET+ | 0, 0, 0, 0.13, 0.03, 0, 5.7 | p < 0.005 |
| PR−RET− | 0, 0, 0, 0 | p < 0.001 |

### Example 4

This Example presents methods for manufacturing light-controllable minicells. A suitable minicell strain is chosen. An example of one such strain is RP437 (Genotype: thr(Am)1, leuB6, his-4, metF(Am)159, eda-50, rpsL1356, thi-1, ara-14, mtl-1, xyl-5, tonA31, tsx-78, lacY1, F2) which has been modified to carry an additional minicell-producing mutation, e.g., ΔminC. As described elsewhere in the specification, other minicell-producing mutations may be used (see, e.g., U.S. patent application Ser. No. 10/154,951, published Oct. 16, 2003 as US20030194798). By overexpressing the FlhD and FlhC proteins (which regulate flagellar synthesis) in this strain, the fraction of motile minicells can be increased from 2% to about 50%, i.e., about half of the minicells produced have at least one working flagellar motor and can swim. Recombinant proteorhodopsin can be expressed from a plasmid. For example, the proteorhodopsin sequence from Beja et al. (Science, (2000) 289:1902-1906; GenBank accession number AF279106, complement (50866..51615) can be used. This sequence can be synthesized or purchased from GeneArt, Inc. (Toronto, Canada) and cloned it any of several commercially available expression vectors (e.g., pBAD, pET161, pET200, pTrc, pTrc2). In addition, the insertion of a gene encoding T7 Polymerase into the parent of the minicell may allow high levels of protein to be produced from T7 promoters upon induction by IPTG. Alternatively, proteorhodopsin could be expressed constitutively using an appropriate constitutive promoter system.

The bacteria are grown in T-broth (0.1% Tryptone, 0.5% NaCl, pH 7-8) at 37° C. with aeration in sterile flasks or 14 ml culture tubes and diluted 1:100 from overnight cultures into fresh broth. Bacteria harboring plasmids are grown in LB supplemented with corresponding antibiotics at standard strengths (e.g., ampicillin (50 μg/mL); kanamycin (25 μg/mL); chloramphenicol (25 μg/mL)).

Strains containing plasmids with inducible proteorhodopsin are induced with 1 mM IPTG or 0.2% arabinose during mid-log growth. At the same time that IPTG or arabinose is added, proteorhodopsin-expressing strains are supplemented with all-trans-retinal to 10 μM. To create fully autonomous genome-less light-responsive recombinant light-harvesting microbes, a plasmid may be employed such as pACCAR16ΔcrtX plasmid [1, 2] (which encodes a carotenogenic gene cluster) augmented with a gene encoding apocarotenoid-15,15′-oxygenase (ACO) [3]. Such a plasmid contains all the genes needed for E. coli to synthesize retinal and parent bacteria containing this plasmid would produce recombinant light-harvesting microbes with functional proteorhodopsin.

When bacterial cultures reach an optical density OD=0.5-1.0, minicells are purified from their parents using filtration through 1.0 μm pore filters composed of polyether-sulfone (Puradisc 25). Filtration is performed Finally, the purified minicells are concentrated by centrifugation at 4500 g for 10 minutes, followed by resuspension in motility buffer using 1-2% original volume. This method yields a population of minicells of which approximately 30% are motile.

Among other features of the light-controllable entities of the invention, the Examples above show that, in certain embodiments, the cellular pmf may be altered to the extent that it both powers cell motility and increases cell survival. The light-controllable entities of the invention can thus withstand respiratory poisons and oxygen depletion via light harvesting. PR expression combined with modulation of the cell's natural pmf generating mechanisms can thus be used to manufacture a diverse array of light-controlled bacteria for a variety of purposes, as described herein.

### Example 5

A photosynthetic S. cerevisae expressing recombinant proteorhodopsin (PR) has been characterized. Cells were illuminated with green LEDs and grown anaerobically. When illuminated, the cells produce 60-100% more ethanol per unit input sugar than PR-cells. Consistent with their increased conversion of input carbon into ethanol, they also produced less CO2 per unit input carbon than PR− cells.

### Example 6

This Example shows that the expression of certain PR derivatives in yeast leads to ultrastructural changes in the cell, including the formation of “membrane stacks,” or multiple layers of membrane folded back on itself, which are highly enriched for PR and reminiscent of plant chloroplasts. A synthetic light-harvesting structure was constructed by expressing a modified form of the proteorhodopsin light-powered proton pump. Proteorhodopsin constructs containing the native bacterial localization signal and fused to a nonmonomeric green-fluorescent protein tag were expressed in S. cerevisae. Expression of the modified PR construct resulted in the formation of multi-lamellar semicircular membrane shelves, as visualized by fluorescence and electron microscopy (FIG. 6). The ability of the PR-nonmonomeric GFP construct to create membrane stacks in S. cerevisae is hypothesized to be the result of protein dimerization or oligomerization; when nonmonomeric GFP is replaced with a strictly monomeric GFP, the membrane stacks disappear.

Alternatively, other membrane structures besides membrane stacks which may be useful in increasing the ability of the cell to pump protons may be created by expressing other PR derivatives. Proteorhodopsin constructs containing the native bacterial localization signal and appended with a small-molecule dimerizer domain were expressed in S. cerevisae. Small-molecule domains dimerize in the presence of a small molecule which is added to the growth medium. When a small molecule was added to the growth medium for the PR-derivative-expressing S. cerevisae, it resulted in the formation of membranes in an architecture resembling a bundle of grapes.

It is proposed that S. cerevisae and other organisms expressing PR derivatives that create additional membrane structures, such as the PR derivatives described herein, will exhibit an increased ability to pump protons in response to illumination, due to the increased effective cross-section and light collection that the membrane shelves provide. It is contemplated that other modifications may be made to proteorhodopsin constructs or other domains or motifs that promote dimerization and/or oligomerization may be appended onto proteorhodopsin constructs in order to create additional membrane structures in organisms expressing these constructs, thereby resulting in the increased ability of those organisms to pump protons in response to illumination. It is also contemplated that domains or motifs having self-affinity, including but not limited to leucine zippers, may be appended onto proteorhodopsin constructs in order to create additional membrane structures in organisms expressing these constructs, thereby resulting in the increased ability of those organisms to pump protons in response to illumination.

The above Examples are provided to illustrate the invention but not to limit its scope. Other variants of the invention will be readily apparent to one of ordinary skill in the art and are encompassed by the appended claims. All publications, databases, Genbank sequences, patents, and patent applications cited herein are hereby incorporated by reference, as well as the claims as filed in this and all priority applications.

What is claimed is:

1. An isolated light-harvesting entity comprising at least one cellular surface, wherein said surface comprises (i) one or more heterologous light-powered proton pumps and (ii) a molecular machine, wherein said molecular machine is directly or indirectly powered by proton motive force, and wherein said one or more heterologous light-powered proton pumps provide at least 5% of the proton motive force in said entity.

2. The entity of claim 1, wherein said entity is a modified prokaryotic or eukaryotic cell.

3. The entity of claim 2, wherein said entity is a modified Escherichia coli cell.

4. The entity of claim 2, wherein said entity is a modified Saccharomyces cerevisiae cell.

5. The entity of claim 1, wherein at least one of said heterologous light-powered proton pumps comprises retinal.

6. The entity of claim 1, wherein at least one of said heterologous light-powered proton pumps is selected from the group consisting of proteorhodopsin and xanthorhodopsin.

7. The entity of claim 1, wherein said molecular machine is a flagellar motor, a rotary ATP synthase, a secretory system, an ion or metabolite anti-porter or symporter, or a multidrug transporter.

8. The entity of claim 2, wherein the one or more heterologous proton pumps provide at least 20% of the total cellular pmf.

9. The entity of claim 2, wherein at least one gene encoding a heterologous light-powered proton pump has been modified to reduce the rate of translation of said encoded heterologous light-powered proton pump relative to an unmodified version of said gene; and wherein the proportion of properly folded, membrane-inserted light-powered proton pumps to improperly folded proton pumps in said entity is increased relative to the proportion comprised by an otherwise identical entity expressing an unmodified version of said gene.

10. The entity of claim 2, wherein the heterologous proton pump is proteorhodopsin, and wherein the immediate C-terminal region of the native proteorhodopsin sequence has been deleted.

11. The entity of claim 2, wherein said entity comprises a non-native stacked membrane structure, wherein said membrane structure comprises said heterologous light-powered proton pump.

12. The entity of claim 11, wherein said modified cell is a modified S. cerevisiae cell.

13. The entity of claim 2, wherein said entity contains light-powered proton pumps in at least two membranes, such as the plasma membrane, the inner mitochondrial membrane, or a synthetic organelle.

14. The entity of claim 13, wherein said membranes are selected from the group consisting of the plasma membrane, the inner mitochondrial membrane, and a synthetic organelle.

15. A system for light-boosted conversion of biomass into biofuels, comprising
(a) a light-collecting entity of claim 1;
(b) a liquid medium, wherein said medium covers said entity;
(c) a light-source.

16. A method for light-enhanced conversion of biomass into a biofuel, comprising the steps of
depositing an entity of claim 1 on a surface or into a liquid containing a carbon source; and
illuminating said entity, resulting in the light-enhanced production of a biofuel, where the fractional conversion enhancement due to illumination is at least 10%, and more typically, 60-100%.

17. A system for effecting light-controlled drug-resistance, comprising
(a) a light-collecting entity of claim 1, wherein said entity comprises a light powered proton pump in one or more cellular compartments;
(b) a light-source.

18. The system of claim 17, wherein said at least one cellular compartment is selected from the group consisting of the vacuole, the ER, Golgi apparatus, and plasma membrane.

19. A method for controlling drug-resistance of a cell using light, comprising (i) depositing an entity of claim 1 onto a surface or into a liquid; and (ii) modulating drug transport by said entity by exposing said entity to light of a particular wavelength, intensity or both.

20. A system for modifying a surface, comprising
(a) a surface to be modified, wherein said surface is doped with a chemical that is toxic to unilluminated cells;
(b) a light-controlled entity of claim 1, wherein said entity is positioned on said surface;
(c) a liquid medium, wherein said medium covers at least a portion of said surface, and wherein said portion includes said entity;
(d) a source for emitting light; and
(e) a controller for manipulating at least one parameter of the light emitted from said source, wherein said parameter is selected from the group consisting of intensity, frequency, and location with respect to said surface.

21. A method for synthesizing patterns on a surface, comprising the steps of placing an entity of claim 1 on a surface, covering at least the portion of said surface to be modified and said entity with a liquid medium, and directing one or more actions of said entity with a light controller, wherein said actions are selected from the group consisting of movement, secretion and chemical degradation.

22. A method for synthesizing a composition in a liquid medium, comprising the steps of placing an entity of claim 1 in a liquid medium, and directing one or more actions of said entity with a light controller, wherein said actions are selected from the group consisting of movement, secretion and chemical degradation.

23. A method for enhancing the ability of an isolated light-harvesting entity to pump protons in response to illumination, comprising expressing in the light-harvesting entity at least one gene encoding a heterologous light-powered proton pump, said gene having been modified to promote the formation of a membrane structure having a plurality of layers of a membrane in said light-harvesting entity.

24. The method of claim 23, wherein said entity is a modified prokaryotic or eukaryotic cell.

25. The method of claim 24, wherein said entity is a modified Escherichia coli cell.

26. The method of claim 24, wherein said entity is a modified Saccharomyces cerevisiae cell.

27. The method of claim 23, wherein said membrane structure comprises said heterologous light-powered proton pump.

28. The method of claim 23, wherein said heterologous light-powered proton pump comprises a proteorhodopsin polypeptide fused to a non-proteorhodopsin polypeptide that promotes protein dimerization or oligomerization.

29. The method of claim 28, wherein said non-proteorhodopsin polypeptide comprises a nonmonomeric fluorescent protein tag.

30. The method of claim 29, wherein said non-proteorhodopsin polypeptide comprises a nonmonomeric green fluorescent protein tag.
